(19) United States
(12) Patent Application Publication
(10) Pub. No.: US 2026/0160765 A1
(43) Pub. Date: Jun. 11, 2026

(54) COMPOSITIONS AND METHODS FOR CHARACTERIZING AND TREATING DISEASES AND DISORDERS ASSOCIATED WITH MULTIPLE ORGAN FAILURE

(71) Applicant: Arizona Board of Regents on Behalf of the University of Arizona, Tucson, AZ (US)

(72) Inventor: Floyd Chilton, Tucson, AZ (US)

(21) Appl. No.: 19/129,204

(22) PCT Filed: Nov. 15, 2023
(86) PCT No.: PCT/US2023/079749

§ 371 Date: May 12, 2025

Related U.S. Application Data
(60) Provisional application No. 63/425,375, filed on Nov. 15, 2022.

(51) Int. Cl. G01N 33/573; G01N 33/68

(52) U.S. Cl. CPC G01N 33/573; G01N 33/6863; G01N 2333/918; G01N 2800/26

(57) ABSTRACT

Provided herein are biomarkers for screening and monitoring of conditions, diseases, and disorders. In particular, provided herein are secreted PLA2 (e.g., sPLA2-IIA) and associated biomarkers for use in characterizing, prognosing, and treating disorders associated with temporal changes in sPLA2 levels.

### CROSS-REFERENCE TO RELATED APPLICATION

This application claims priority to U.S. provisional patent application Ser. No. 63/425,375, filed Nov. 15, 2022, which is incorporated herein by reference in its entirety.

### STATEMENT REGARDING FEDERALLY SPONSORED RESEARCH OR DEVELOPMENT

This invention was supported by Grant No. R01AT008621 awarded by the National Institutes of Health. The government has certain rights in the invention.

### FIELD OF THE INVENTION

Provided herein are biomarkers for screening and monitoring of conditions, diseases, and disorders. In particular, provided herein are secreted PLA2 (e.g., sPLA2-IIA) and associated biomarkers for use in characterizing, prognosing, and treating disorders associated with temporal changes in sPLA2 levels.

### BACKGROUND

COVID-19 is a respiratory condition caused by the SARS-COV-2 coronavirus. Some people are infected but don't notice any symptoms. Most people will have mild symptoms and get better on their own. But about 1 in 6 will have severe problems, such as trouble breathing. The odds of more serious symptoms are higher for older patients or those with another health condition like diabetes or heart disease.

Common symptoms include fever, fatigue, a dry cough, loss of appetite, body aches, shortness of breath, and mucus or phlegm. COVID-19 is typically diagnosed via a PCR test on a nasal or throat swab.

Severity is initially classified by respiratory factors such as respiration rate, oxygen saturation and pulmonary lesion progression and more critical cases are often complicated by organ dysfunction, including septic shock, heart failure and disseminated intravascular coagulation.

Most people diagnosed with COVOD-19 do not require medical treatment. Severe cases are treated with oxygen nasal cannulas or a ventilator.

Drugs used to treat COVID-19 include the anti-viral drug remdesivir, blood thinners such as heparin, monoclonal antibodies including casirivimab and imdevimab, convalescent plasma, and dexamethasone. There is no specific therapy for severe COVID-19.

As new highly transmissible and potential vaccine-evading SARS-COV-2 strains emerge with increasing disease-associated mortality and morbidity, there is an urgent need to elucidate central molecular and cellular mechanisms that limit host fitness in severe and lethal COVID-19 cases.

Infection with SARS-COV-2 can also lead to lingering or new symptoms beyond the acute COVID-19 illness, currently termed post-acute sequelae of SARS-COV-2 infection (PASC). In fact, while the exact epidemiology is unknown, as most reports are limited by selection bias and follow up, early reports of prolonged or new symptoms that develop after acute COVID-19 are staggering. Current studies report a high percentage of patients reporting PASC symptoms for weeks to months after the acute infection. In one large international study, 91% of participants reported symptoms longer than 35 weeks. Fatigue/malaise and dyspnea are two of the most common and debilitating symptoms reported, with either still reported in >50% and >37%, respectively at 12 months.

Unfortunately, despite a high percentage of persistently reported dyspnea and fatigue, there are inconsistent associations between objective clinical, laboratory, or radiographic findings and severity of symptoms. As such, determining underlying pathobiological mechanisms to identify potential therapeutic targets are desperately needed.

### SUMMARY

Experiments described herein utilized machine learning methods to determine that secreted PLA2 isoforms are the most critical to determining and stratifying the patients who will ultimately die of multiple organ failure (e.g., from sepsis related to COVID-19). While a one-time measure of the levels of these enzymes provides a basis for understanding the seriousness of these diseases, it is not as accurate as it might be in stratifying the patient is in terms of disease severity and the likelihood of death. Accordingly, provided herein are improved methods of identifying and treating subjects likely to have severe disease and/or multiple organ failure.

For example, in some embodiments, provided herein is a method of treating a condition, disease or disorder in a subject, comprising: a) assaying a first sample from the subject for the level of a secreted phospholipase A2 (sPLA2) at a first time point; b) assaying a second sample from the subject for the level of a secreted phospholipase A2 (sPLA2) at a second time point; c) repeating step b) at least one time at one or more third time points; and d) administering potentially lifesaving therapeutics (e.g., an sPLA2 inhibitor and/or a corticosteroid) to the subject when the level of sPLA2 increases or decreases from the first time point to the third time point.

Also provided is a method of diagnosing or providing a prognosis of a severe or lethal condition, disease or disorder in a subject, comprising: a) assaying a first sample from the subject for the level of a secreted phospholipase A2 (sPLA2) at a first time point; b) assaying a second sample from the subject for the level of a secreted phospholipase A2 (sPLA2) at a second time point; c) repeating step b) at least one time at one or more third time points; and d) identifying the subject as likely to develop severe disease or die when the level of sPLA2 increases or decreases from the first time point to the third time point.

Further provided herein is a method of assaying a sample from a subject with a condition, disease or disorder in a subject, comprising: a) assaying a first sample from the subject for the level of a secreted phospholipase A2 (sPLA2) at a first time point; b) assaying a second sample from the subject for the level of a secreted phospholipase A2 (sPLA2) at a second time point; and c) repeating step b) at least one time at one or more third time points.

In some embodiments, the method further comprises repeating the assaying step one or more times before and/or after said administering step.

The present disclosure is not limited to a particular sPLA2. Examples include but are not limited to, sPLA₂-IB, sPLA₂-IIA, sPLA₂-IIC, sPLA₂-II-D, sPLA₂-II-E, sPLA₂-II-F, sPLA₂-III, sPLA2-V, sPLA₂-X, sPLA₂-XIIA, or sPLA₂-XIIB. In some embodiments, the sPLA₂ is sPLA₂-IIA. In some embodiments, the increase or decrease is an increase in the level of sPLA₂-IIA, sPLA₂-V, sPLA₂-X, sPLA₂-IB, sPLA₂-IIC, or sPLA₂-XVI or a decrease in the level of sPLA₂-IID or sPLA₂-XIIB. In some embodiments, an increase in the level of one more sPLA2 enzymes selected sPLA₂-IIA, sPLA₂-V, sPLA₂-X, sPLA₂-IB, sPLA₂-IIC, or sPLA₂-XVI is indicative of an increased risk of death or severe disease. In some embodiments, a decrease in the level of one more sPLA2 enzymes selected from sPLA₂-IID or sPLA₂-XIIB is indicative of an increased risk of death or severe disease. In some embodiments, a decrease in level of sPLA₂-IIA, sPLA₂-V, sPLA₂-X, sPLA₂-IB, sPLA₂-IIC, or sPLA₂-XVI and/or an increase in the level of sPLA₂-IID or sPLA₂-XIIB from the second to third time point is indicative of severe disease that is likely to resolve.

The present disclosure is not limited to a particular interval between the first, second, and one or more third time points. In some embodiments, the first, second and third time points are daily, weekly, monthly, or yearly. In some embodiments, the first, second and third time points are day 0, 3, and 7 (e.g., after a subject is admitted to the hospital or emergency room). In some embodiments, a machine learning algorithm is used to generate a decision tree that stratifies the subject into likelihood of developing mild disease, severe disease, or death.

The present disclosure is not limited to a particular condition, disease, or disorder. In some embodiments, the disorder is or includes multiple organ failure. Examples include but are not limited to, a respiratory disorder (ARDS), a trauma, a bacterial infection, septic shock, heart failure, a bite from a venomous snake, or disseminated intravascular coagulation.

In some embodiments, the subject is infected with or has been infected with the SARS-CoV-2 virus. In some embodiments, the subject has one or more symptoms of COVID-19. Certain subjects are at increased risk of severe disease or death from said condition, disease or disorder (e.g., by being over the age of 65 or having one or more additional risk factors for severe disease).

Further embodiments comprise assaying one or more of the subject's respiration rate, oxygen saturation or pulmonary lesion progression. In some embodiments, assays for the level of sPLA₂ comprises an immunoassay. In some embodiments, the sample is blood or a blood product.

The present disclosure is not limited to particular sPLA₂ inhibitors. Examples include but are not limited to, a nucleic acid (e.g., an antisense nucleic acid, a miRNA, an siRNA, or an shRNA), an antibody, or a small molecule (e.g., varespladib methyl, AZD2716, 7,7-Dimethyleicosadienoic Acid (DEDA), oleyloxyethyl phosphorylcholine, luffariellolide, thioetheramide PC, 4-[(1-oxo-7-phenylheptyl)amino]-(4R)-octanoic acid, LY315920, or YS-[(1-oxo-7-phenylheptyl)amino]-4-(phenylmethoxy)-benzenepentanoic acid). In some embodiments, the small molecule is a corticosteroid (e.g., dexamethasone). In some embodiments, the inhibitor is an sPLA₂-XIIB polypeptide.

Additional embodiments are described herein.

### BRIEF DESCRIPTION OF THE DRAWINGS

FIG. 1A-D: Secreted PLA2 Family, Distribution of Clinical Acuity Categories of COVID-19 Cohort and Volcano Plots Showing Baseline Fold Change Comparisons between Acuity Groups. A. A phylogenetic tree of sPLA₂ family and representative functions of each isoform. B. Distribution of the COVID-19-infected cohort with, day 0, day 3, and day 7 timepoints used in downstream proteomic analyses. Categories referenced in later analyses are defined as Death, Severe, and Mild for A1, A2, and (A3-A4), respectively. C. Volcano plot displaying p-values of Bonferroni-adjusted, two-sided independent t-tests and average fold-changes between the baseline (day 0) protein levels of combined Death+Severe vs. Mild-category patients, and D Death vs. Severe-category patients.

FIG. 2: Kinetics of Levels of sPLA₂ Isoforms in Different Severity Groups. Log₂ Relative fluorescence units (RFUs) of sPLA₂ isoform levels by severity over time. Asterisks denote the significance of differences between two days' values using two-sided independent t-tests.

FIG. 3A-C: Category Classification Using Changes in sPLA₂-IIA Levels. A. Spaghetti plot for longitudinal log, protein levels of sPLA₂-IIA isoform separated by death, severe, and mild categories for 128 patients with all 3 timepoints. B. Decision tree stratifying patients from the death, severe, and mild categories. C. ROC curves and corresponding AUC values reflect the performance of decision trees generated to classify each category alone against the combination of the others (binary classification).

FIG. 4: Involvement of the sPLA₂ Isoforms in COVID-19. The current study shows a persistent increase in sPLA₂-IIA,-IIC,-V, and-X in deceased patients and lower levels in milder categories. In contrast, the levels of sPLA₂-IID and-XIIB decrease with lethality and are elevated in milder categories.

FIG. 5: Kinetics of sPLA₂-V and sPLA-X isoform levels. Spaghetti plots for longitudinallog2 protein levels of sPLA₂-V and sPLA₂-X isoforms separated by death, severe, and mild categories for 214 patients.

FIG. 6: PLA2G2A kinetics as reference and kinetics of 5 chemokines ordered by baseline (day 0) fold change magnitude between death+severe vs. mild baseline values.

### DEFINITIONS

To facilitate an understanding of the present invention, a number of terms and phrases are defined below:

As used herein, the terms “detect”, “detecting” or “detection” may describe either the general act of discovering or discerning or the specific observation of a metabolite.

As used herein, the term “subject” refers to any organisms that are screened using the methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, tumors, (e.g., biopsy samples), cells, and gases. Biological samples include blood products, such as plasma, serum and the like. Such examples are not however to be construed as limiting the sample types applicable to the present invention. A “reference level” of an analyte means a level of the analyte (e.g., sPLA₂) that is indicative of a particular disease state, phenotype, or lack thereof, as well as combinations of disease states, phenotypes, or lack thereof. A “positive” reference level of an analyte means a level that is indicative of a particular disease state or phenotype. A “negative” reference level of an analyte means a level that is indicative of a lack of a particular disease state or phenotype.

A “reference level” of a metabolite may be an absolute or relative amount or concentration of the analyte, a presence or absence of the analyte, a range of amount or concentration of the analyte, a minimum and/or maximum amount or concentration of the analyte, a mean amount or concentration of the analyte, and/or a median amount or concentration of the analyte. Appropriate positive and negative reference levels of an analyte for a particular disease state, phenotype, or lack thereof may be determined by measuring levels of the analyte in one or more appropriate subjects, and such reference levels may be tailored to specific populations of subjects (e.g., a reference level may be age-matched so that comparisons may be made between metabolite levels in samples from subjects of a certain age and reference levels for a particular disease state, phenotype, or lack thereof in a certain age group). Such reference levels may also be tailored to specific techniques that are used to measure levels of the analyte in biological samples (e.g., LC-MS, GC-MS, etc.), where the levels of the analyte may differ based on the specific technique that is used.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

“Mass Spectrometry” (MS) is a technique for measuring and analyzing molecules that involves fragmenting a target molecule, then analyzing the fragments, based on their mass/charge ratios, to produce a mass spectrum that serves as a “molecular fingerprint”.

Determining the mass/charge ratio of an object is done through means of determining the wavelengths at which electromagnetic energy is absorbed by that object. There are several commonly used methods to determine the mass to charge ration of an ion, some measuring the interaction of the ion trajectory with electromagnetic waves, others measuring the time an ion takes to travel a given distance, or a combination of both. The data from these fragment mass measurements can be searched against databases to obtain definitive identifications of target molecules. Mass spectrometry is also widely used in other areas of chemistry, like petrochemistry or pharmaceutical quality control, among many others.

### DETAILED DESCRIPTION OF THE INVENTION

Bacteremia, Sepsis, ARDS are highly heterogenous serious clinical conditions where outcomes depend on factors beyond the signs and symptoms and include age, infection source, and the appropriateness and timing of therapeutic interventions. There is currently a shift in the research from merely predicting outcomes to using pathobiological understandings to reveal how the human host will respond to these conditions and how to use this information to improve clinical outcomes.

Experiments described herein utilized machine learning methods to determine that secreted PLA2 isoforms are the most critical to determining and stratifying the patients who will ultimately die of multiple organ failure (e.g., from sepsis related to COVID-19). While a one-time measure of the levels of these enzymes provides a basis for understanding the seriousness of these diseases, it is not as accurate as it might be in stratifying the patient is in terms of disease severity and the likelihood of death.

The current disclosure reveals that a temporal analysis of the levels of these enzymes accurately predicts patient outcomes and particularly determining the patients who are recovering and resolving their condition and those who mowing toward a more serious and lethal outcome. This in turn facilities the selection and timing of therapeutic interventions. Additionally, a highly active and potential lethal component within snake venom is also a sPLA₂ isoform and a temporal analysis of levels of these enzymes determines the status of patients and also stratifies them for selection and timing of therapeutic interventions for snake bites.

The compositions and methods described herein provide a point of use, point of care diagnostic test in patients with a number of conditions associated with multiple organ failure (e.g., including but not limited to, bacteremia, sepsis, ARDS or snake bites) to determine the clinical direction of patients with regard to catastrophic outcomes. In some embodiments, the test is provided in the doctor's office, ER and/or ICU. In some embodiments, assay methods described herein are utilized with immunosuppressed patients (e.g., those undergoing chemotherapy or bone marrow transplant) to detect potentially lethal infections before the source of those infection can be determined.

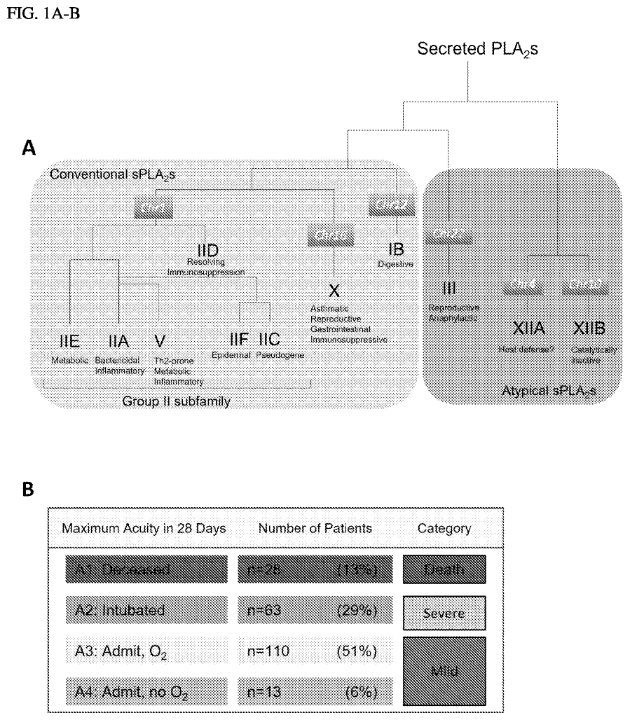

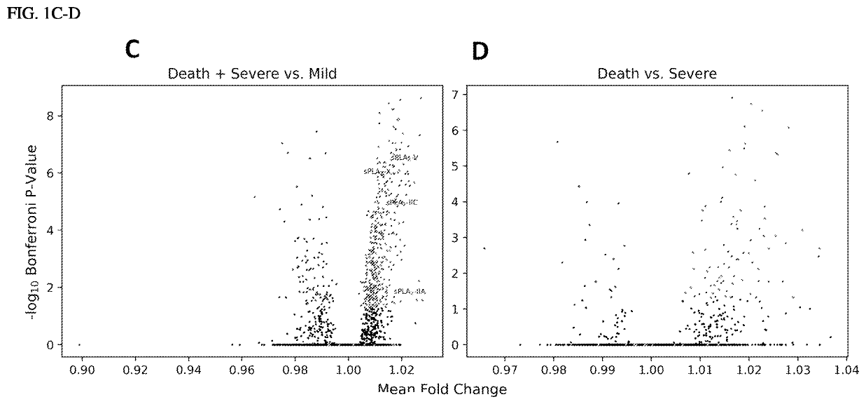

The secreted PLA2 (sPLA₂) family includes low molecular weight proteins (14-15 kDa) that require millimolar Ca²⁺ for their catalytic activity and primarily hydrolyze target phospholipids in the extracellular space or on the outer layers of cellular membranes (8, 9). The mammalian sPLA₂ family presently contains 10 known catalytically active isoforms (FIG. 1A; sPLA₂-IB, sPLA₂-IIA, sPLA₂-IIC, sPLA₂-II-D, sPLA₂-II-E, sPLA₂-II-F, sPLA₂-III, sPLA₂-V, sPLA₂-X, sPLA₂-XIIA) and one catalytically inactive isoform (sPLA₂-XIIB) with distinct tissue/cellular distributions, phospholipid substrate specificities, and diverse biology.

Experimental and clinical studies of sPLA₂ are described in reference 10-14. Experiments described herein used machine learning algorithms to identify sPLA₂-IIA as the key variable among 80 clinical indices with the capacity to stratify severe COVID-19 survivors from deceased victims. Absence of temporal kinetics of sPLA₂-IIA levels limited the previous study, as did lack of sPLA₂ isoform selective ELISA kits to detect and quantify other sPLA₂ isoforms. The current study addresses both limitations using the clinical dataset of Filbin et al (15) and plasma proteomics analysis of eight sPLA₂ isoforms and an intracellular calcium-dependent PLA₂ (16, 17) collected on days 0, 3, and 7 of patients entering the ICU. Temporal correlations of several sPLA₂ isoforms with COVID-19 deaths recapitulated earlier sepsis studies. Furthermore, differences in the kinetics of sPLA₂ isoforms known to be proinflammatory versus immunosuppressive also emphasized the potential divergent roles sPLA₂ family members in COVID-19 severity.

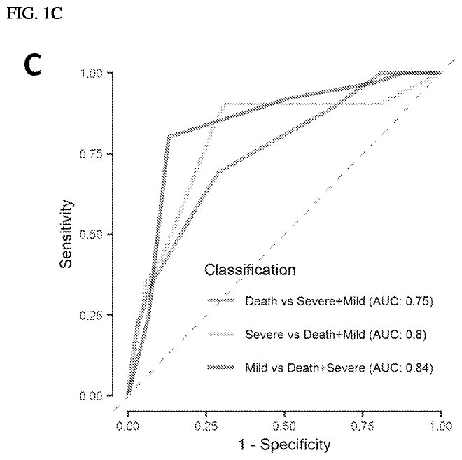

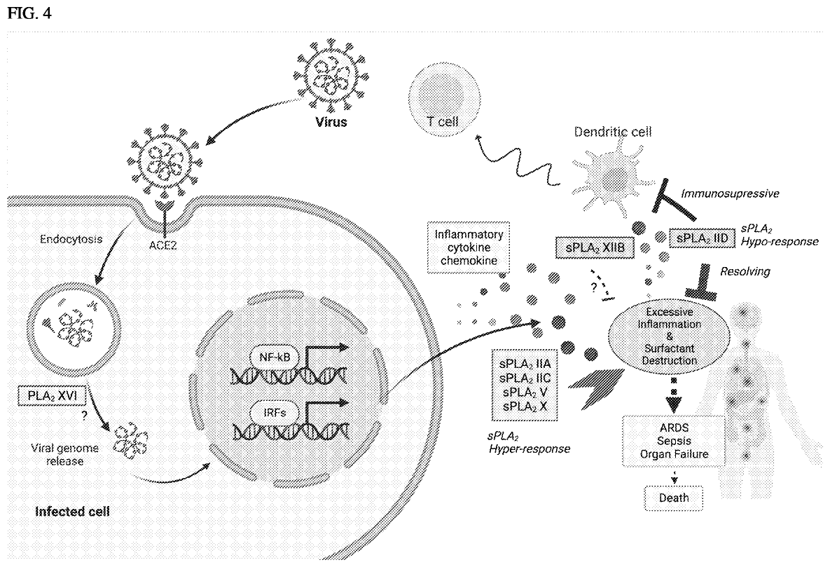

Experiments described herein examined sPLA₂ kinetics and expanded the contributions of sPLA₂ isoforms to COVID-19 disease pathobiology and prognosis. The plasma levels of several sPLA₂ isoforms, including sPLA₂-IIA, sPLA₂-X, sPLA₂-V, sPLA₂-IB, and sPLA₂-XVI, taper in severe and surviving patients, whereas levels continued to increase in non-survivors. A machine learning analysis (decision tree) confirmed that the kinetic behavior of PLA2-IIA can separate patients into their respective acuity categories. This study also identifies that other isoforms sPLA₂-V, sPLA₂-X, sPLA₂ IB, and sPLA₂-XVI may underlie lethal mechanisms associated with COVID-19 disease or other disease and conditions that ultimately lead to pulmonary surfactant damage and deadly organ failure (FIG. 4). sPLA₂-V expression in the lung is elevated in asthma patients and asthma mice models, where it localizes to epithelial cells, mast cells, and alveolar macrophages (20-23). Pla2g5−/− knockout mice are protected from alveolar injury after antigen, LPS, or ventilator challenge, suggesting a key role in lung inflammation (24, 25). sPLA₂-V also may promote antigen presentation to T cells and hydrolyze pulmonary surfactant (26-28).

Because of sPLA₂-X's affinity for phosphatidylcholine and arachidonic acid, much of its proinflammatory properties has been attributed to arachidonic acid derived mediators. sPLA₂-X occurs in airway epithelial and mast cells and it hydrolyzes pulmonary surfactant (22). Pla2 g10--mice are protected against antigen-induced asthma showing marked reductions in eosinophils, smooth muscle layer thickening, and eicosanoid biosynthesis (20, 22, 29, 30), and these asthmatic responses are restored by a knock-in of the human PLA2G10 gene (30). sPLA₂-X has pro-tumorigenic activity in B cell lymphoma via its capacity to hydrolyze highly unsaturated fatty acids such as docosahexaenoic acid from extracellular vesicles; these fatty acids in turn are converted to oxylipin metabolites that promote tumor growth (31).

Experiments described herein also identified time dependent reductions in two sPLA₂ isoforms, sPLA₂-IID and sPLA₂-XIIB, in severe COVID-19 disease, with overall higher levels in milder patients. sPLA₂-IID is a “resolving sPLA₂ isoform” (FIG. 4) that mobilizes omega-3 highly unsaturated fatty acids, such as docosahexaenoic acid, that then can serve as precursors for anti-inflammatory/pro-resolving metabolites, such as resolvin D1 (32-36). There is an age-dependent increase in this isoform that may worsen outcomes in mice infected with the SARS-COV-2 virus (34). Middle-aged knockout mice lacking expression of PLA2G2D have less severe disease and death from SARS-COV-2 (37). sPLA₂-IID is constitutively expressed in dendritic cells and represses immune responses in The1, Th2 and Th17 suppressing anti-viral and anti-tumor activities (36).

Little is known about secreted sPLA₂-XIIB except that it is catalytically inactive due to altered phospholipid binding properties, may regulate HNF4alpha-induced infectivity of hepatitis C, and is down-regulated in cancer (38-42). sPLA₂-XIIB kinetics mirror that of sPLA₂-IID and this supports that it may also have an immunosuppressive role in COVID-19 disease and other inflammatory diseases (FIG. 4). PLA2-XVI is not a member of the sPLA₂ family and, as a low molecular weight intracellular protein, has phospholipase A1, PLA2, and acyl transferase activities. This low molecular weight PLA2 is thought to play a role in enabling Picornaviridae infections by facilitating virion-mediated transfer into the cytoplasm (17). The higher levels and time-dependent increase of this enzyme in deceased and severe patients supports a role in SARS-COV-2 infectivity.

For example, in some embodiments, provided herein is a method of treating a condition, disease or disorder in a subject, comprising: a) assaying a first sample from the subject for the level of a secreted phospholipase A2 (sPLA₂) at a first time point; b) assaying a second sample from the subject for the level of a secreted phospholipase A2 (sPLA₂) at a second time point; c) repeating step b) at least one time at one or more third time points; and d) administering an sPLA₂ inhibitor to the subject when the level of sPLA₂ increases or decreases from the first time point to the third time point.

Also provided is a method of diagnosing or providing a prognosis of a severe or lethal condition, disease or disorder in a subject, comprising: a) assaying a first sample from the subject for the level of a secreted phospholipase A2 (sPLA₂) at a first time point; b) assaying a second sample from the subject for the level of a secreted phospholipase A2 (sPLA₂) at a second time point; c) repeating step b) at least one time at one or more third time points; and d) identifying the subject as likely to develop severe disease or die when the level of sPLA₂ increases or decreases from the first time point to the third time point.

Further provided herein is a method of assaying a sample from a subject with a condition, disease or disorder in a subject, comprising: a) assaying a first sample from the subject for the level of a secreted phospholipase A2 (sPLA₂) at a first time point; b) assaying a second sample from the subject for the level of a secreted phospholipase A2 (sPLA₂) at a second time point; and c) repeating step b) at least one time at one or more third time points.

The present disclosure is not limited to a particular sPLA₂. Examples include but are not limited to, sPLA₂-IB, sPLA₂-IIA, sPLA₂-IIC, sPLA₂-II-D, sPLA₂-II-E, sPLA₂-II-F, sPLA₂-III, sPLA₂-V, sPLA₂-X, sPLA₂-XIIA, or sPLA₂-XIIB. In some embodiments, the sPLA₂ is sPLA₂-IIA. In some embodiments, the increase or decrease is an increase in the level of sPLA₂-IIA, sPLA₂-V, sPLA₂-X, sPLA₂-IB, sPLA₂-IIC, or sPLA₂-XVI or a decrease in the level of sPLA₂-IID or sPLA₂-XIIB. In some embodiments, an increase in the level of one more sPLA2 enzymes selected sPLA₂-IIA, sPLA₂-V, sPLA₂-X, sPLA₂-IB, sPLA₂-IIC, or sPLA₂-XVI is indicative of an increased risk of death or severe disease. In some embodiments, a decrease in the level of one more sPLA2 enzymes selected from sPLA₂-IID or sPLA₂-XIIB is indicative of an increased risk of death or severe disease. In some embodiments, a decrease in level of sPLA₂-IIA, sPLA₂-V, sPLA₂-X, sPLA₂-IB, sPLA₂-IIC, or sPLA₂-XVI and/or an increase in the level of sPLA₂-IID or sPLA₂-XIIB from the second to third time point is indicative of severe disease that is likely to resolve.

The present disclosure is not limited to a particular interval between the first, second, and one or more third time points. In some embodiments, the first, second and third time points are daily, weekly, monthly, or yearly. In some embodiments, the first time point (day 0) is the day a subject is admitted to a hospital or ICU. In some embodiments, the second and third time points are day 3 and day 7. In some embodiments, a fourth time point is day 9 or 10.

In some embodiments, the sample is blood or a blood product (e.g., plasma). In some embodiments, the method further comprises assaying one or more of the subject's respiration rate, oxygen saturation or pulmonary lesion progression.

The present disclosure is not limited to a particular method of assaying the level of sPLA₂-IIA. Examples include, but are not limited to, immunoassays (e.g., ELISA assays, fluorescent immunoassay, chemiluminescent immunoassay, radioimmunoassay, colorimetric enzyme activity assay, and fluorometric enzyme activity assay.

Any suitable sPLA2 (e.g., sPLA2-IIA) inhibitor may be utilized in the methods described herein. Examples include but are not limited to, antibodies, nucleic acids (e.g., antisense nucleic acids, siRNAs, shRNAs, miRNAs, etc.), and small molecules (e.g., varespladib methyl, AZD2716, 7,7-Dimethyleicosadienoic Acid (DEDA), oleyloxyethyl phosphorylcholine, luffaricllolide, thioctheramide PC, 4-[(1-oxo-7-phenylheptyl)amino]-(4R)-octanoic acid, LY315920, or YS-[(1-oxo-7-phenylheptyl)amino]-4-(phenylmethoxy)-benzenepentanoic acid). In some embodiments, the small molecule is a corticosteroid (e.g., dexamethasone). In some embodiments, the inhibitor is an sPLA2-XIIB polypeptide.

The present disclosure is not limited to particular conditions, diseases, or disorders. Examples include but are not limited to, a respiratory disorder, a trauma, a bacterial infection, septic shock, heart failure, or disseminated intravascular coagulation. In some embodiments, the respiratory disorder is or includes acute respiratory distress syndrome (ARDS). In some embodiments, the subject is infected with or has been infected with the SARS-COV-2 virus. In some embodiments, the subject has one or more symptoms of COVID-19.

The compositions and methods described herein find use in treating both acute COVID-19 and post-COVID-19 syndrome (e.g., persistent fatigue and/or muscle and nerve dysfunction following an active COVID-19 infection). In some embodiments, the assaying is repeated at one or more time points (e.g., weekly, monthly, or yearly). In some embodiments, the administering is continued until the level of sPLA2-IIA drops below the threshold level and/or the subject has a decrease in symptoms of post-COVID-19 syndrome or COVID-19.

In some embodiments, the subject is at increased risk of severe disease or death from the disorder (e.g., due to age over 65 or comorbidities).

The level of sPLA2-IIA is compared to reference levels using various techniques, including a simple comparison (e.g., a manual comparison) of the level(s) of sPLA2-IIA in the biological sample to reference levels (e.g., the level in a subject not diagnosed with a respiratory disorder). The level may also be compared to reference levels using one or more statistical analyses (e.g., t-test, Welch's T-test, Wilcoxon's rank sum test, random forest).

The present disclosure is not limited to a particular reference level of sPLA2-IIA. In some embodiments, the elevated level of sPLA2-IIA is an elevated level relative to a reference level selected from the group consisting of the level in a subject not diagnosed with a respiratory disorder, the level of the subject prior to being diagnosed with the respiratory disorder, and a population average of subjects not diagnosed with respiratory disorders. In some embodiments, the elevated level of sPLA2-IIA is above 10, 20, 30, 40, 50, 100, 150, 200, 250, 300, 350, or 400 ng/ml. In some embodiments, the patient also has a blood urea nitrogen (BUN) level greater than or equal to 16 mg/dl.

Any patient sample suspected of containing sPLA2-IIA is tested according to the methods described herein. By way of non-limiting examples, the sample may be blood, urine, or a fraction thereof (e.g., plasma, serum, urine supernatant, urine cell pellet, urine sediment, or prostate cells).

In some embodiments, the patient sample undergoes preliminary processing designed to isolate or enrich the sample for sPLA2-IIA. A variety of techniques may be used for this purpose, including but not limited: centrifugation; immunocapture; and cell lysis.

sPLA2-IIA may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized.

In other embodiments, sPLA2-IIA is detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable method may be used to analyze the biological sample in order to determine the presence, absence or level(s) of sPLA2-IIA. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

In some embodiments, a computer-based analysis program (e.g., machine learning algorithm) is used to translate the raw data generated by the detection assay (e.g., the presence, absence, or amount of a sPLA2) into data of predictive value for a clinician. In some embodiments, the analysis program utilizes a machine learning algorithm (e.g., to generate a decision tree).

The clinician can access the predictive data using any suitable means. Thus, in some embodiments, the present invention provides the further benefit that the clinician, who is not likely to be trained in analysis or the use of machine learning algorithms, need not understand the raw data. The data is presented directly to the clinician in its most useful form. The clinician is then able to immediately utilize the information in order to optimize the care of the subject. The present invention contemplates any method capable of receiving, processing, and transmitting the information to and from laboratories conducting the assays, information provides, medical personal, and subjects. For example, in some embodiments of the present invention, a sample (e.g., a blood, urine or plasma sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., sPLA2-IIA level), specific for the diagnostic or prognostic information desired for the subject.

The profile data is then prepared in a format suitable for interpretation by a treating clinician. For example, rather than providing raw data, the prepared format may represent a diagnosis or risk assessment (e.g., prediction of the severity of respiratory disease) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

When the amount(s) or level(s) of sPLA2-IIA in the sample are determined, the amount(s) or level(s) may be compared to reference levels, such as the levels in healthy individuals to aid in diagnosing or to diagnose whether the subject has severe disease. Levels of the one or more metabolites in a sample corresponding to the reference levels (e.g., levels that are the same as the reference levels, substantially the same as the reference levels indicative of severe disease, above and/or below the minimum and/or maximum of the reference levels, and/or within the range of the reference levels) are indicative of a diagnosis, risk, or prognosis of severe disease in the subject. Levels of the one or more metabolites in a sample corresponding to reference levels below the level associated with severe disease (e.g., levels that are the same as the reference levels, substantially the same as the reference levels, above and/or below the minimum and/or maximum of the reference levels, and/or within the range of the reference levels) are indicative of a diagnosis of mild or moderate disease in the subject.

In some embodiments, quantitative reference levels for a specific diagnosis or prognosis are determined and utilized to provide a risk assessment, diagnosis, prognosis, or treatment.

### Experimental

### Example 1

### Methods

### Dataset

Proteomic and clinical index datasets generated by Filbin et al. were obtained from Mendeley Data (15). The datasets included 384 patients enrolled from the Emergency Department (ED) of the Massachusetts General Hospital (MGH) with 306 patients positive for SARS-COV-2. Patient acuity levels were classified (A1-A5) based on the World Health Organization (WHO) Ordinal Outcomes Scale and determined by their severest condition within 28 days post-enrollment. Among the 306 COVID patients, 214 had blood samples obtained at two time points (days 0 and 3) and 128 at three timepoints (days 0, 3, and 7). Plasma levels of 5,201 proteins were measured using the SomaScan platform and expressed as Relative Fluorescence Units (RFUs). Our reanalysis included the 214 COVID patients with at least two blood samples, of which 28 patients died (A1), 63 required intubations (A2), 110 required supplemental oxygen (A3), and 13 were without supplemental oxygen during hospitalization (A4). Patients discharged from ED without hospitalization and blood samples at day 3 (A5) were not included in our reanalysis. A1, A2, and (A3-A4) were grouped into the categories of death (n=28), severe (n=63), and mild (n=123), respectively. Protein level RFUs were log 2 transformed. A1 and 42 values were calculated as the differences between log 2 protein levels on days 3 and 0 and days 7 and 3, respectively.

### Statistics

Significance of protein level differences between sample groups were calculated using independent two-sided t-tests with Bonferroni correction to correct for multiple hypothesis testing. Adjusted p-values were binned into 0-0.0001, 0.0001-0.001, 0.001-0.01, 0.01-0.05, and 0.05-1 and labeled in plots with four to zero asterisks, respectively.

### Decision Tree Analysis

A classification model used the A1 and 42 values of sPLA2-IIA through recursive partitioning (decision tree) to classify patient categories (death, severe, mild) using the R package rpart (18). The minimum number of samples at a node to allow a split was set to 20 and the complexity assessed by the lowest 10-fold cross-validation error. Patients with missing 42 values were not propagated through decisions involving Δ2 (use surrogate=0). Splits calculations used Gini impurity. Performance assessed using ROCs and AUCs were calculated using the pROC (19) package.

### Results

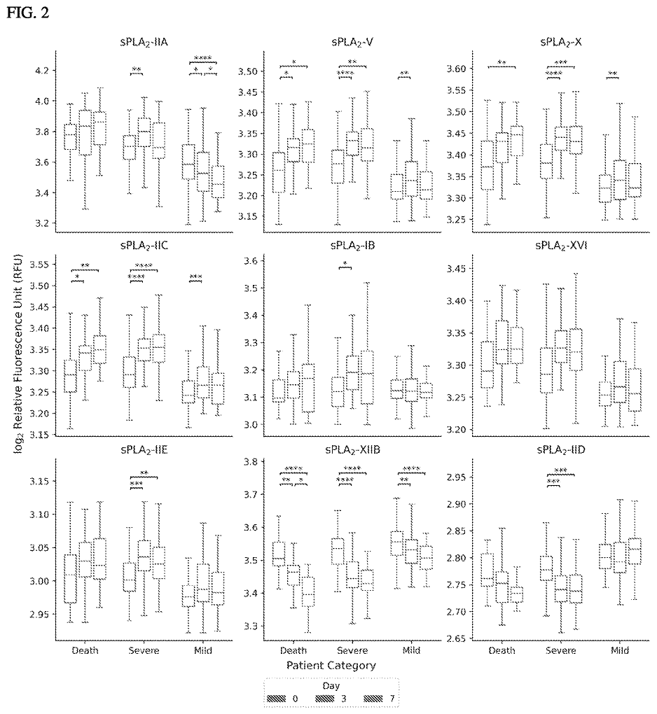

FIG. 1B shows the four clinical primary acuity levels of 214 COVID-19 patients with day 0, day 3, and, when available, day 7 timepoints used in downstream proteomic analyses. FIG. 1C compares fold changes and statistical significance of measured proteins in deceased patients+intubated patients versus milder disease. Among 492 statistically significant elevated proteins, sPLA2-IIA, sPLA2-V, sPLA2-X and sPLA2-IIC ranked 8th, 227th, 231st, and 337th, respectively. FIG. 1D shows that, when assessing only baseline levels, none of these sPLA2 isoforms significantly differed between patients who died compared to severe or intubated. FIG. 2 compares levels of the nine measurable PLA2 isoforms including sPLA2-IB, sPLA2-IIA, sPLA2-IIC, sPLA2-IID, sPLA2-IIE, sPLA2-V, sPLA2-X, sPLA2-XIIB, and PLA2-XVI. Six of the nine isoforms, sPLA2-IIA, sPLA2-V, sPLA2-X, sPLA2-IB, sPLA2-IIC, and PLA2-XVI, increased over time in non survivors. In contrast, ventilated survivors begin to resolve elevations between day 3 and day 7. Mild patients had reduced levels of sPLA2-IIA, sPLA2-IIC, sPLA2-IIE, sPLA2-V, sPLA2-X, and PLA2-XVI over time. In contrast, there was a time-dependent decrease in sPLA2-IID and sPLA2-XIIB in patients who died and in severe patients who lived. These two isoforms also were found at higher levels in less severe patients.

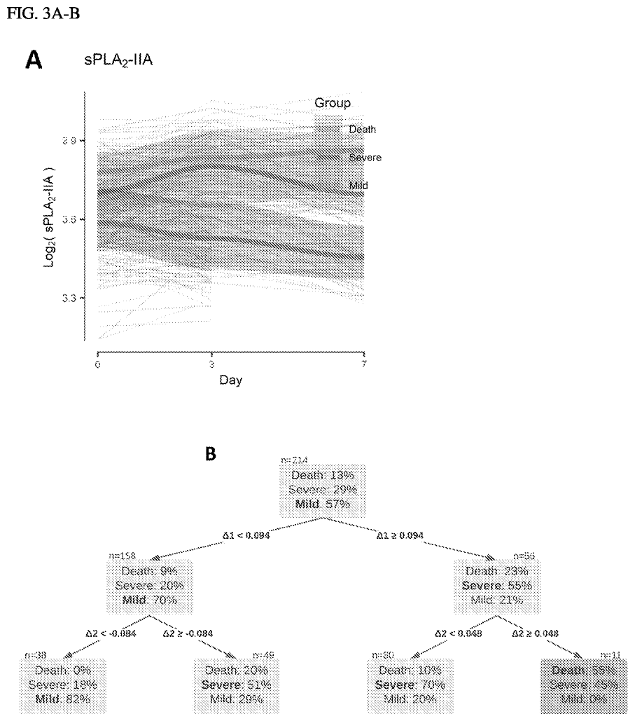

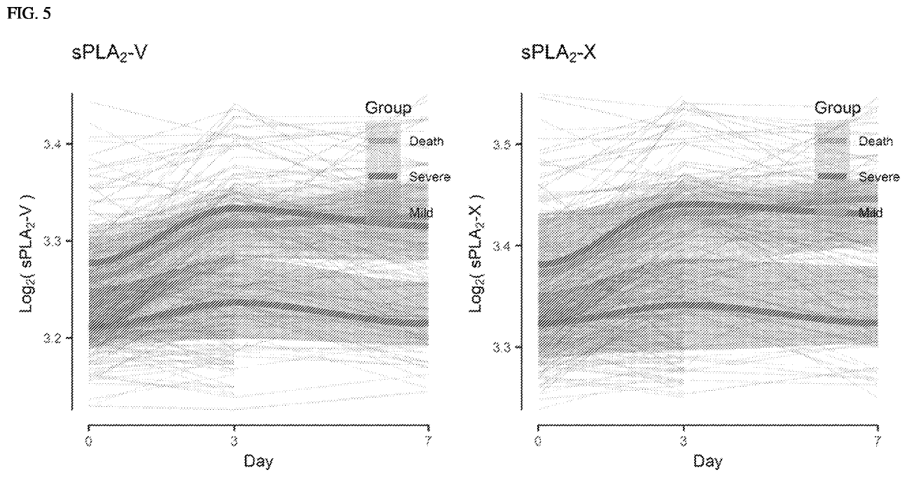

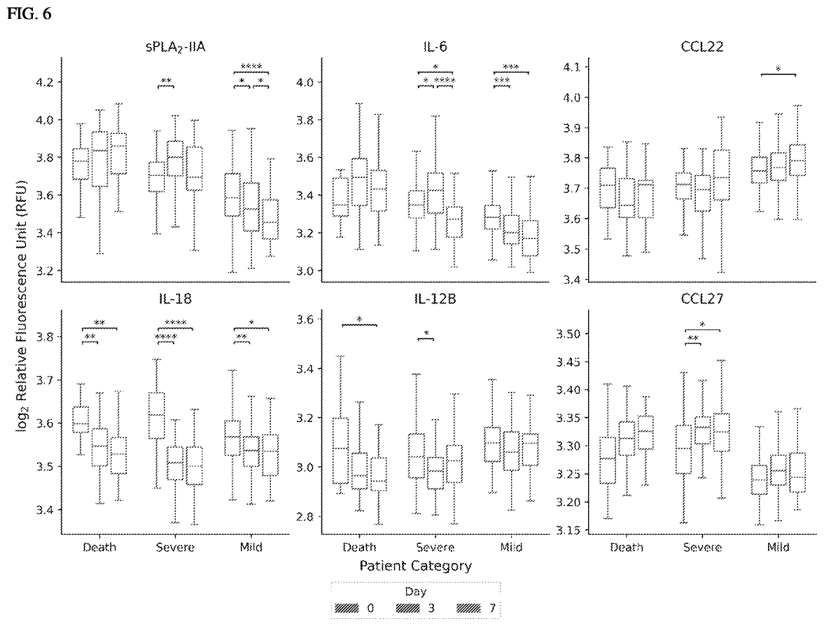

FIG. 3A is a spaghetti plot of the longitudinal flow of sPLA2-IIA and confirms PLA2-IIA as a key protein associated with patient outcome. FIG. 5 shows spaghetti plots for both sPLA2-V and sPLA2-X, which follow a similar pattern of PLA2-IIA. A decision tree generated by recursive partitioning assessed whether changes in PLA2-IIA levels between day 0 and day 3 (41) and between day 3 and day 7 (42) could stratify patients into mild, severe, and deceased categories (FIG. 3B). The decision tree corroborated the sPLA2-IIA kinetics by first separating the death and severe categories from the mild category using A1 values. Patients with a moderate increase in PLA2-IIA from day 0 to day 3 (A1≥0.094) were at a significantly higher risk of death and intubation. Furthermore, a continuous increase in PLA2-IIA from day 3 to day 7 (42≥ 0.048) further enriched for death versus severe but survived (right-most branch). In comparison, a further decrease in PLA2-IIA from day 3 to day 7 (42<0.048) enriched for the mild patient category as depicted on the left-most branch. Tree performance was assessed by classifying each single acuity group (death, severe, or mild) against the combination of the other groups. The corresponding AUCs (area under the ROC curve=0.75, 0.8, 0.84) suggest that the kinetics of PLA2-IIA alone may distinguish between death, severe, and mild disease categories (FIG. 3C). FIG. 6 compares the kinetics of sPLA2-IIA with several plasma cytokines and chemokines. These cytokines and chemokines had the highest-ranking baseline fold changes between the intubated patients+deceased patients versus the milder patient categories. In contrast to sPLA2-IIA, IL-6, IL-18, and IL-12B median levels were decreased between day 3 and day 7 in deceased patients while CCL22 kinetics show an unclear trend with time and patient severity. A time-dependent increase in the chemokine CCL27 occurred in deceased patients with CCL27 leveling in both intubated and mild categories, suggesting a similar trend to sPLA2-IIA. Table 1 lists recursive partitioning classifiers using A1 and 42 values of the selected cytokines, chemokines, and sPLA2 isoforms, all of which performed worse than sPLA2-IIA using the same optimization procedure to identify the minimum 10-fold cross-validation error rate.

TABLE 1

| | Minimum Cross |
|---|---|
| Protein | Validation Error |
| sPLA₂-IIA | 0.30 |
| IL-6 | 0.35 |
| CCL22 | 0.425 |
| IL-18 | 0.39 |
| IL-12B | 0.425 |
| CCL27 | 0.425 |
| sPLA₂-V | 0.425 |
| sPLA₂-X | 0.425 |
| sPLA₂-IIC | 0.425 |
| sPLA₂-IB | 0.425 |
| sPLA₂-IIE | 0.425 |
| sPLA₂-XVI | 0.425 |
| sPLA₂-XIIB | 0.31 |
| sPLA₂-IID | 0.37 |

### REFERENCES

1. Gupta A, Madhavan M V, Schgal K, Nair N, Mahajan S, Schrawat T S, ct al. Extrapulmonary manifestations of COVID-19. Nat Med. 2020; 26 (7): 1017-32.
2. Guan W J, Ni Z Y, Hu Y, Liang W H, Ou C Q, He J X, et al. Clinical Characteristics of Coronavirus Disease 2019 in China. N Engl J Med. 2020; 382 (18): 1708-20.
3. Bansal R, Gubbi S, Muniyappa R. Metabolic Syndrome and COVID 19: Endocrine-Immune-Vascular Interactions Shapes Clinical Course. Endocrinology. 2020; 161 (10).
4. Umemura Y, Yamakawa K, Kiguchi T, Nishida T, Kawada M, Fujimi S. Hematological phenotype of COVID-19-induced coagulopathy: far from typical sepsis-induced coagulopathy. J Clin Med. 2020; 9 (9).
5. Guo T, Fan Y, Chen M, Wu X, Zhang L, He T, et al. Cardiovascular implications of fatal outcomes of patients with coronavirus disease 2019 (COVID-19). JAMA Cardiol. 2020; 5 (7): 811-8.
6. Shen B, Yi X, Sun Y, Bi X, Du J, Zhang C, et al. Proteomic and metabolomic characterization of COVID-19 patient sera. Cell. 2020; 182 (1): 59-72.e15.
7. Wu D, Shu T, Yang X, Song J-X, Zhang M, Yao C, et al. Plasma metabolomic and lipidomic alterations associated with COVID-19. Natl Sci Rev. 2020; 7 (7): 1157-68.
8. Murakami M, Nakatani Y, Atsumi G I, Inoue K, Kudo I. Regulatory Functions of Phospholipase A2. Crit Rev Immunol. 2017; 37 (2-6): 127-95.
9. Murakami M, Sato H, Taketomi Y. Updating phospholipase A (2) biology. Biomolecules. 2020; 10 (10).
10. Vadas P. Elevated plasma phospholipase A2 levels: correlation with the hemodynamic and pulmonary changes in gram-negative septic shock. J Lab Clin Med. 1984; 104 (6): 873-81.
11. Nyman K M, Uhl W, Forsström J, Büchler M, Beger H G, Nevalainen T J. Serum phospholipase A2 in patients with multiple organ failure. J Surg Res. 1996; 60 (1): 7-14.
12. Guidet B, Piot O, Masliah J, Barakett V, Maury E, Bereziat G, et al. Secretory non-pancreatic phopholipase A2 in severe sepsis: relation to endotoxin, cytokines and thromboxane B2. Infection. 1996; 24 (2): 103-8.
13. Vadas P, Pruzanski W, Farewell V. A predictive model for the clearance of soluble phospholipase A2 during septic shock. J Lab Clin Med. 1991; 118 (5): 471-5.
14. Snider J M, You J K, Wang X, Snider A J, Hallmark B, Zec M M, et al. Group IIA secreted phospholipase A2 is associated with the pathobiology leading to COVID-19 mortality. J Clin Invest. 2021; 131 (19): e149236.
15. Filbin M R, Mehta A, Schneider A M, Kays K R, Guess J R, Gentili M, et al. Longitudinal proteomic analysis of severe COVID-19 reveals survival-associated signatures, tissue-specific cell death, and cell-cell interactions. Cell Rep Med. 2021; 2 (5): 100287.
16. Jaworski K, Ahmadian M, Duncan R E, Sarkadi-Nagy E, Varady K A, Hellerstein M K, et al. AdPLA ablation increases lipolysis and prevents obesity induced by high-fat feeding or leptin deficiency. Nat Med. 2009; 15 (2): 159-68.
17. Zádori Z, Szelei J, Lacoste M C, Li Y, Gariépy S, Raymond P, et al. A viral phospholipase A2 is required for parvovirus infectivity. Dev Cell. 2001; 1 (2): 291-302.
18. Therneau T, Atkinson B, Ripley B. Recursive partitioning for classification, regression and survival trees. R package version 4.1.16 [Internet]. 2022; The Comprehensive R Archive Network. Available from: https://cran.r-project.org/web/packages/rpart/index.html.
19. Robin X, Turck N, Hainard A, Tiberti N, Lisacek F, Sanchez J C, et al. pROC: an open-source package for R and S+ to analyze and compare ROC curves. BMC Bioinformatics. 2011; 12:77.
20. Murakami M, Yamamoto K, Miki Y, Murase R, Sato H, Taketomi Y. Chapter Four-The Roles of the Secreted Phospholipase A2 Gene Family in Immunology. In: Alt F W, editor. Advances in Immunology. 132. Academic Press; 2016. p. 91-134.
21. Muñoz NM, Meliton A Y, Arm J P, Bonventre J V, Cho W, Leff A R. Deletion of secretory group V phospholipase A2 attenuates cell migration and airway hyperresponsiveness in immunosensitized mice. J Immunol. 2007; 179 (7): 4800-7.
22. Seeds M C, Jones K A, Duncan Hite R, Willingham M C, Borgerink H M, Woodruff R D, et al. Cell-specific expression of group X and group V secretory phospholipases A (2) in human lung airway epithelial cells. Am J Respir Cell Mol Biol. 2000; 23 (1): 37-44.
23. Triggiani M, Giannattasio G, Calabrese C, Loffredo S, Granata F, Fiorello A, et al. Lung mast cells are a source of secreted phospholipases A2. J Allergy Clin Immunol. 2009; 124 (3): 558-65, 65.e1-3.
24. Muñoz NM, Meliton A Y, Meliton L N, Dudek S M, Leff A R. Secretory group V phospholipase A2 regulates acute lung injury and neutrophilic inflammation caused by LPS in mice. Am J Physiol Lung Cell Mol Physiol. 2009; 296 (6): L879-87.
25. Giannattasio G, Fujioka D, Xing W, Katz H R, Boyce J A, Balestrieri B. Group V secretory phospholipase A2 reveals its role in house dust mite-induced allergic pulmonary inflammation by regulation of dendritic cell function. J Immunol. 2010; 185 (7): 4430-8.
26. Henderson W R, Jr., Ye X, Lai Y, Ni Z, Bollinger J G, Tien Y T, et al. Key role of group v secreted phospholipase A2 in Th2 cytokine and dendritic cell-driven airway hyperresponsiveness and remodeling. PLOS One. 2013; 8 (2): e56172.
27. Meliton A Y, Muñoz NM, Meliton L N, Birukova A A, Leff A R, Birukov K G. Mechanical induction of group V phospholipase A (2) causes lung inflammation and acute lung injury. Am J Physiol Lung Cell Mol Physiol. 2013; 304 (10): L689-700.
28. Ohta S, Imamura M, Xing W, Boyce J A, Balestrieri B. Group V secretory phospholipase A2 is involved in macrophage activation and is sufficient for macrophage effector functions in allergic pulmonary inflammation. J Immunol. 2013; 190 (12): 5927-38.
29. Henderson W R, Jr., Chi E Y, Bollinger J G, Tien Y T, Ye X, Castelli L, et al. Importance of group X-secreted phospholipase A2 in allergen-induced airway inflammation and remodeling in a mouse asthma model. J Exp Med. 2007; 204 (4): 865-77.
30. Henderson W R, Jr., Oslund R C, Bollinger J G, Ye X, Tien Y T, Xue J, et al. Blockade of human group X secreted phospholipase A2 (GX-sPLA2)-induced airway inflammation and hyperresponsiveness in a mouse asthma model by a selective GX-sPLA2 inhibitor. J Biol Chem. 2011; 286 (32): 28049-55.
31. Kudo K, Miki Y, Carreras J, Nakayama S, Nakamoto Y, I to M, et al. Secreted phospholipase A (2) modifies extracellular vesicles and accelerates B cell lymphoma. Cell Metab. 2022; 34 (4): 615-33.e8.
32. Miki Y, Yamamoto K, Taketomi Y, Sato H, Shimo K, Kobayashi T, et al. Lymphoid tissue phospholipase A2 group IID resolves contact hypersensitivity by driving antiinflammatory lipid mediators. J Exp Med. 2013; 210 (6): 1217-34.
33. Miki Y, Kidoguchi Y, Sato M, Taketomi Y, Taya C, Muramatsu K, et al. Dual roles of group IID phospholipase A2 in inflammation and cancer. J Biol Chem. 2016; 291 (30): 15588-601.
34. Vijay R, Hua X, Meyerholz D K, Miki Y, Yamamoto K, Gelb M, et al. Critical role of phospholipase A2 group IID in age-related susceptibility to severe acute respiratory syndrome-CoV infection. J Exp Med. 2015; 212 (11): 1851-68.
35. Sato H, Taketomi Y, Miki Y, Murase R, Yamamoto K, Murakami M. Secreted phospholipase PLA2G2D contributes to metabolic health by mobilizing @3 polyunsaturated fatty acids in WAT. Cell Rep. 2020; 31 (5): 107579.
36. Murakami M, Miki Y, Sato H, Murase R, Taketomi Y, Yamamoto K. Group IID, IIE, IIF and III secreted phospholipase A (2) s. Biochim Biophys Acta Mol Cell Biol Lipids. 2019; 1864 (6): 803-18.
37. Wong L R, Zheng J, Wilhelmsen K, Li K, Ortiz M E, Schnicker N J, et al. Eicosanoid signalling blockade protects middle-aged mice from severe COVID-19. Nature. 2022; 605 (7908): 146-51.
38. Liu Q, Yang M, Fu X, Liu R, Sun C, Pan H, et al. Activation of farnesoid X receptor promotes triglycerides lowering by suppressing phospholipase A2 G12B expression. Mol Cell Endocrinol. 2016; 436:93-101.
39. Gelb M H, Valentin E, Ghomashchi F, Lazdunski M, Lambeau G. Cloning and recombinant expression of a structurally novel human secreted phospholipase A2. J Biol Chem. 2000; 275 (51): 39823-6.
40. Rouault M, Bollinger J G, Lazdunski M, Gelb M H, Lambeau G. Novel mammalian group XII secreted phospholipase A2 lacking enzymatic activity. Biochemistry. 2003; 42 (39): 11494-503.
41. Murakami M, Masuda S, Shimbara S, Bezzine S, Lazdunski M, Lambeau G, et al. Cellular arachidonate-releasing function of novel classes of secretory phospholipase A2s (groups III and XII). J Biol Chem. 2003; 278 (12): 10657-67.
42. Li X, Jiang H, Qu L, Yao W, Cai H, Chen L, et al. Hepatocyte nuclear factor 4α and downstream secreted phospholipase A2 GXIIB regulate production of infectious hepatitis C virus. J Virol. 2014; 88 (1): 612-27.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of treating a condition, disease or disorder in a subject, comprising:
a) assaying a first sample from said subject for the level of a secreted phospholipase A2 (sPLA2) at a first time point;
b) assaying a second sample from said subject for the level of a secreted phospholipase A2 (sPLA2) at a second time point;
c) repeating step b) at least one time at one or more third time points; and
d) administering an sPLA2 inhibitor and/or a corticosteroid to said subject when the level of sPLA2 increases or decreases from said first time point to said third time point.

2. A method of diagnosing a severe or lethal condition, disease or disorder in a subject, comprising:
a) assaying a first sample from said subject for the level of a secreted phospholipase A2 (sPLA2) at a first time point;
b) assaying a second sample from said subject for the level of a secreted phospholipase A2 (sPLA2) at a second time point;
c) repeating step b) at least one time at one or more third time points; and
d) identifying said subject as likely to develop severe disease or die when the level of sPLA2 increases or decreases from said first time point to said third time point.

3. A method of assaying a sample from a subject with a condition, disease or disorder in a subject, comprising:
a) assaying a first sample from said subject for the level of a secreted phospholipase A2 (sPLA2) at a first time point;
b) assaying a second sample from said subject for the level of a secreted phospholipase A2 (sPLA2) at a second time point; and
c) repeating step b) at least one time at one or more third time points.

4. The method of any of the preceding claims, further comprising repeating said assaying step one or more times before and/or after said administering step.

5. The method of any of the preceding claims, wherein said time points are day 0, day 3, and day 7.

6. The method of claim 5, wherein said day 0 is the day said subject is admitted to the hospital or emergency room.

7. The method of any of the preceding claims, said sPLA2 is selected from the group consisting of sPLA₂-IB, sPLA₂-IIA, sPLA₂-IIC, sPLA₂-II-D, sPLA₂-II-E, sPLA₂-II-F, sPLA₂-III, sPLA₂-V, sPLA₂-X, sPLA₂-XIIA, and sPLA₂-XIIB.

8. The method of any of the preceding claims, wherein said sPLA₂ is sPLA₂-IIA.

9. The method of any of any of the preceding claims, wherein said increase or decrease is an increase in the level of sPLA₂-IIA, sPLA₂-V, sPLA₂-X, sPLA₂-IB, sPLA₂-IIC, and sPLA₂-XVI or a decrease in the level of sPLA₂-IID or sPLA₂-XIIB.

10. The method of any of any of the preceding claims, wherein an increase in the level of one more sPLA₂ enzymes selected from the group consisting of sPLA₂-IIA, sPLA₂-V, sPLA₂-X, sPLA₂-IB, sPLA₂-IIC, or sPLA₂-XVI is indicative of an increased risk of death or severe disease.

11. The method of any of any of the preceding claims, wherein a decrease in the level of one more sPLA₂ enzymes selected from the group consisting of sPLA₂-IID and sPLA₂-XIIB is indicative of an increased risk of death or severe disease.

12. The method of any of any of the preceding claims, wherein a decrease in level of sPLA₂-IIA, sPLA₂-V, sPLA₂-X, sPLA₂-IB, sPLA₂-IIC, or sPLA₂-XVI and/or an increase in the level of sPLA₂-IID and sPLA₂-XIIB from the second to third time point is indicative of severe disease that is likely to resolve.

13. The method of any of the preceding claims, wherein a machine learning algorithm is used to generate a decision tree that stratifies said subject into likelihood of developing mild disease, severe disease, or death.

14. The method of any of the preceding claims, wherein said inhibitor is an sPLA₂-XIIB polypeptide.

15. The method of any of the preceding claims, wherein said condition, disease, or disorder is or includes multiple organ failure.

16. The method of any of the preceding claims, wherein said condition, disease or disorder is selected from the group consisting of a respiratory disorder, a trauma, a bacterial infection, septic shock, heart failure, a bite from a venomous snake, and disseminated intravascular coagulation.

17. The method of claim 16, wherein said respiratory disorder is acute respiratory distress syndrome (ARDS).

18. The method of any of the preceding claims, wherein said subject is infected with or has been infected with the SARS-COV-2 virus.

19. The method of claim 18, wherein said subject has one or more symptoms of COVID-19.

20. The method of any of the preceding claims, wherein said subject is at increased risk of severe disease or death from said condition, disease or disorder.

21. The method of any of the preceding claims, wherein said subject is over the age of 65.

22. The method of any of the preceding claims, further comprising assaying one or more of the subject's respiration rate, oxygen saturation or pulmonary lesion progression.

23. The method of any of the preceding claims, wherein said assaying comprises an immunoassay.

24. The method of any of the preceding claims, wherein said sPLA₂ inhibitor is selected from the group consisting of a nucleic acid, an antibody, and a small molecule.

25. The method of claim 24, wherein said small molecule is selected from the group consisting of varespladib methyl, AZD2716, 7,7-Dimethyleicosadienoic Acid (DEDA), oleyloxyethyl phosphorylcholine, luffariellolide, thioetheramide PC, 4-[(1-oxo-7-phenylheptyl)amino]-(4R)-octanoic acid, LY315920, and yS-[(1-oxo-7-phenylheptyl)amino]-4-(phenylmethoxy)-benzenepentanoic acid.

26. The method of claim 24, wherein said small molecule is a corticosteroid.

27. The method of claim 26, wherein said corticosteroid is dexamethasone.

28. The method of claim 24, wherein said nucleic acid is selected from the group consisting of an antisense nucleic acid, a miRNA, an siRNA, and an shRNA.

29. The method of any of the preceding claims, wherein said sample is blood or a blood product.

30. The method of any of the preceding claims, wherein said first, second and third time points are daily, weekly, monthly, or yearly.
